(19) United States
(12) Patent Application Publication
(10) Pub. No.: US 2006/0020310 A1
(43) Pub. Date: Jan. 26, 2006

(54) Apparatus for sonophotodynamic therapy

(75) Inventors: Nicolas G. Loebel, Redmond, WA (US); Roy Wallace Martin, Anacortes, WA (US); Andreas Rose, Sammamish, WA (US)

Correspondence Address: DOBRUSIN & THENNISCH PC, 29 W LAWRENCE ST, SUITE 210, PONTIAC, MI, 48342, US

(73) Assignee: Ondine International Ltd.

(21) Appl. No.: 11/144,433

(22) Filed: Jun. 3, 2005

Related U.S. Application Data
(60) Provisional application No. 60/590,421, filed on Jul. 22, 2004.
(60) Provisional application No. 60/622,463, filed on Oct. 27, 2004.

(51) Int. Cl. A61N 5/06; A61C 1/07

(52) U.S. Cl. 607/89; 433/119

(57) ABSTRACT

As apparatus for providing sonophotodynamic therapy includes one or more members in communication with one or more of the following: a sonic energy source; a therapeutic fluid source and a light source.

### CLAIM OF BENEFIT OF FILING DATE

This application claims the benefit of U.S. Provisional Application Ser. No. 60/590,421 titled: “Dental Photocidal Therapy by Means of Dental Scalers” filed on Jul. 22, 2004 and U.S. Provisional Application Ser. No. 60/622,463 titled: “Improved Dental Scaler for Use in Photocidal Therapy” filed on Oct. 27, 2004.

### FIELD OF THE INVENTION

The present invention relates to a single integrated apparatus for performing sonophotodynamic therapy upon tissue of an organism.

### BACKGROUND OF THE INVENTION

Chronic periodontitis, a form of inflammatory periodontal disease, is the major cause of tooth loss in adults. Patients with chronic periodontitis have inflamed pockets in the gum tissue, or gingiva, surrounding the affected tooth. Layers of bacteria build up in biofilm within these gingival pockets, leaving behind calcified accretions called calculus attached to the tooth and root surfaces. As the bacterial infection progresses, inflammatory exudates from the biofilm as well as host tissue responses can cause progressive breakdown of the hard and soft tissue structures supporting the tooth, ultimately resulting in tooth loss. Bacterial infections of the oral cavity are also gaining recognition as a source of infection in the rest of the body (e.g., bacteremias [infections of the blood], infective carditis, pulmonary disease, etc.) Such infections have also been implicated in implant rejection and may complicate the prognosis for diabetes mellitus and other autoimmune disorders.

Conventional methods of treating bacterial infections of the oral cavity include removal of the pockets of subgingival plaque, calculus and biofilms by dental scaling and applications of antibiotics. Dental scaling is performed on patients with periodontal diseases several times a year, in some cases every three months or more frequently. An ultrasonic dental scaler generates ultrasound vibrations in a fluid (e.g., water, saline or the like) that remove subgingival plaque, calculus and biofilm from the gum tissues. The ultrasound vibrations cause cavitation exerting high shear forces directly on the fluid, the calculus, and the plaque surrounding or within the gum tissue, resulting in the detachment of such calculus, plaque and associated biofilm from the gum tissues. The principles of dental scalers are well described in the patent literature. See U.S. Pat. Nos. 2,990,616; 3,089,790; 3,703,037; 3,990,452; 4,283,174; 4,804,364; and 6,619,957. These patents are all hereby incorporated by reference.

Unfortunately, dental scaling by itself has had limited success in eliminating bacteria in the oral cavity and long term applications of antibiotics could lead to resistance rendering the antibiotics clinically ineffective. Moreover, applications of antibiotics may not be desirable for immunocompromised patients and patients with denture stomatitis.

In addition to treatment of inflammatory periodontal diseases, elimination of microbes in the oral cavity is also preferable in drilled out carious cavities prior to conventional filling and during other forms of dental surgery including endodotic operations involving the interior of the tooth itself.

Photodynamic therapy for killing microbes in the oral cavity was disclosed by Wilson, et al. in U.S. Pat. No. 5,611,793 and European Patent No. EP 0637976B2. These patents are herein incorporated by reference. As discussed in these patents, laser light in a certain wavelength and intensity range is used to illuminate a photosensitive compound that has been applied to the infected tissue(s). The laser activates the compound causing the formation of free radicals and other elements that are toxic to microbes residing in the oral cavity.

### SUMMARY OF INVENTION

Because photodynamic therapy has been shown to be effective in killing infectious microbes in the oral cavity, it would be highly desirable if it were incorporated into routine dental care (e.g., dental scaling or the like). It is an objection of the present invention to provide an apparatus that can conveniently and efficiently disinfect a treatment region of the oral cavity while cleaning and removing calculus, plaque and biofilm from such a region.

The commonly owned and copending application titled “Sonophotodynamic Therapy” provides methods that use a photosensitizing composition in conjunction with irradiation by light and/or sonic energy to kill microbes in the oral cavity and wound in other part(s) of the body (i.e., not in the oral cavity), a process hereinafter termed “sonophotodynamic therapy”. This application is hereby incorporated by reference in its entirety for all purposes. As described below, the combined administration of light and sonic energy in the presence of a fluid and a photosensitizing compound has a synergistic effect in the killing of microbes in the oral cavity.

The application of sonic energy in a fluid can create acoustic cavitation. Acoustic cavitation involves the nucleation, growth and collapse of gas/vapor filled bubbles in a fluid. Cavitation can effectively kill microbes by physical disruption. For example, the mechanical energy in acoustic cavitation can disrupt and disperse plaque (and the microbes surrounding it) by the violent shear forces produced around the bubbles. Free radicals in a fluid have also been detected as a direct result of acoustic cavitation. These free radicals can kill microbes via cell wall disruption and/or lipid peroxidation. The collapse of the bubbles during acoustic cavitation can be accompanied by a simultaneous emission of light (“sonoluminescence”). The light emitted by sonoluminescence is very broadband and may contain ultraviolet light, which can also be directly detrimental to microbes. Light emitted via sonoluminescence, when applied to a photosensitizing composition in the oral cavity, can release more free radicals, causing further killing of microbes.

In an aspect of the invention, an apparatus for performing sonophotodynamic therapy is disclosed comprising: a light source; a sonic energy source; a cooling or lavage fluid source; and one or more members in communication with the light source, the sonic. energy source and the cooling or lavage fluid source for respectively providing light, sonic energy and cooling or lavage fluid to tissue of an organism; wherein the one or more members include a single member or a plurality of members integrated together

In another aspect of the invention, an apparatus for performing sonophotodynamic therapy is disclosed comprising: a light source; a sonic energy source; a photosensitizing composition source; and one or more members in communication with the light source, the sonic energy source and the photosensitizing composition source for respectively providing light, sonic energy and cooling or lavage fluid to tissue of an organism; wherein the one or more members include a plurality of members integrated together; and wherein the one or more members include a tube in fluid communication with the photosensitizing composition and a waveguide in communication with the light source, the waveguide being located at least partially within the tube.

In yet another aspect of the invention, an apparatus for performing sonophotodynamic therapy is disclosed comprising: a light source; a sonic energy source; a photosensitizing composition source; a cooling or lavage fluid source; and one or more members in communication with the light source, the sonic energy source, the cooling or lavage fluid source and the photosensitizing composition source for providing ultrasonic energy, light and photosensitizing composition to tissue of an organism, wherein: i. the one or more members include a waveguide for the delivery of light from the light source, a dental scaler for delivery of sonic energy from the sonic energy source, a tube defining a passageway for delivery of the cooling or lavage fluid from the cooling or lavage fluid source and a tube defining a passageway for delivery of photosensitizing composition from the photosensitizing composition source; and wherein the one or more members include a plurality of members integrated together into a single probe.

A further understanding of the nature and advantages of the present invention may be realized by reference to the remaining portion of the specifications and the drawings.

### BRIEF DESCRIPTION OF THE DRAWINGS

The features and inventive aspects of the present invention will become more apparent upon reading the following detailed description, claims, and drawings, of which the following is a brief description:

FIG. 1 illustrates an exemplary apparatus for performing sonophotodynamic therapy in accordance with an aspect of the present invention;

FIG. 2 is a side view of a portion of an exemplary probe suitable for use as part of the apparatus of FIG. 1;

FIGS. 2A-2C illustrate exemplary tips suitable for use with the apparatus of the present invention;

FIG. 3 illustrates a portion of an exemplary insert suitable for use as part of a probe of the apparatus of the present invention;

FIG. 4 illustrates another portion of the exemplary insert of FIG. 3;

FIG. 5 illustrates an exemplary connection of an exemplary probe to the remainder of the apparatus of the present invention;

FIG. 6 shows an alternative exemplary probe suitable for use in the apparatus of the present invention;

FIG. 6A illustrates a cross-section of the probe of FIG. 6 taken along line 6A-6A;

FIG. 7 illustrates an exemplary portion of the exemplary probe of FIG. 6;

FIG. 7A is a sectional cut-away view of the exemplary portion of the exemplary probe of FIG. 7;

FIG. 8 illustrates another exemplary portion of the exemplary probe of FIG. 6;

FIG. 8A illustrates a cross-section of the exemplary portion of FIG. 8 taken along line 8A-8A;

FIG. 8B is a sectional cut-away view of the exemplary portion of the exemplary probe of FIG. 8;

FIG. 9 illustrates another exemplary alternative probe suitable for use in the apparatus of the present invention; and

FIG. 9A is a view of an end of the probe of FIG. 9.

### DESCRIPTION OF THE PREFERRED EMBODIMENT

The present invention is predicated upon the provision of a single integrated apparatus capable of performing sonophotodynamic therapy upon tissue of an organism. Generally, it is contemplated that the apparatus may be employed to perform sonophotodynamic therapy upon any tissue of any organism alive or dead and/or upon objects such as denture or other prosthetics and should not be limited to performing therapy on any particular tissue, organism or other object unless otherwise specifically recited. The apparatus has been found to be particularly useful, however, for performing sonophotodynamic therapy upon tissue within the oral cavities of humans.

### I. DEFINITIONS

The following terms are intended to have the following general meanings as they are used herein.

1. Microbes: any and all disease-related microbes such as virus, fungus, and bacteria including Gram-negative organisms, Gram-positive organisms or the like.

2. Light: light at any wavelengths that can be absorbed by a photosensitizing composition. Such wavelengths include wavelengths selected from the continuous electromagnetic spectrum such as ultraviolet (“UV”), visible, the infrared (near, mid and far), etc. The wavelengths are generally preferably between about 160 nm to 1600 nm, more preferably between 400 nm to 800 nm, most preferably between about 500 nm to 850 nm although the wavelengths may vary depending upon the particular photosensitizing compound used and the light intensity. The light may be produced by any suitable art-disclosed light emitting devices such as lasers, light emitting diodes (“LEDs”), arc lamps, incandescent sources, fluorescent sources, gas discharge tubes, thermal sources, light amplifiers or the like.

3. Locus: any tissue, carious cavity, endodontic chamber, wound, or lesion in the oral cavity where anti-microbial treatment is desired.

4. Wound: any wound or lesion outside of the oral cavity where anti-microbial treatment is desired.

5. Photosensitizing composition: a composition comprising at least one suitable art-disclosed photosensitizer. Arianor steel blue, toluidine blue O, crystal violet, methylene blue and its derivatives, azure blue cert, azure B chloride, azure 2, azure A chloride, azure B tetrafluoroborate, thionin, azure A eosinate, azure B eosinate, azure mix sicc., azure II eosinate, haematoporphyrin HCl, haematoporphyrin ester, aluminium disulphonated phthalocyanine are examples of suitable photosensitizers. Porphyrins, pyrroles, tetrapyrrolic compounds, expanded pyrrolic macrocycles, and their respective derivatives are further examples of suitable photosensitizers. Photofrin® manufactured by QLT PhotoTherapeutics Inc., Vancouver, B.C., Canada is yet another example of a suitable photosensitizer. Other exemplary photosensitizers may be found in U.S. Pat. Nos. 5,611,793 and 6,693,093. U.S. Pat. No. 6,693,093 is hereby incorporated by reference. The photosensitizers mentioned above are examples are not intended to limit the scope of the present invention in any way.

6. Sonic energy: ultrasound, sonic waves or energy produced by a sonic or ultrasonic device (e.g., dental scaler or the like). It is preferred that the tip vibration of the sonic device is between the range of about 3 KHz to about 5 MHz, more preferably between about 10 KHz to about 1 MHz, even more preferably between about 20 KHz to about 50 KHz, and most preferably between about 25 KHz to about 40 KHz.

### II. Exemplary Apparatus for Sonophotodynamic Therapy

### i. Description of the Apparatus

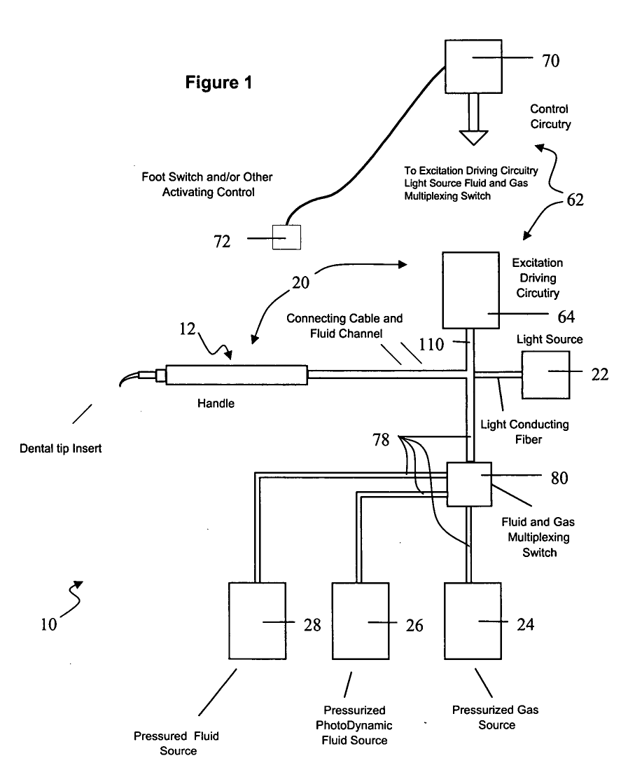

Referring to FIG. 1, there is illustrated one exemplary apparatus 10 capable of performing sonophotodynamic therapy for killing microbes or bacteria located upon or within tissue. The apparatus 10 includes a probe 12 in communication (e.g., fluid communication, electrical communication or light communication) with the one or more of the following components: a sonic energy source 20, a light source 22, a gas source 24; a therapeutic fluid (e.g. a photosensitizing composition) source 26 and a cooling and/or lavage fluid source 28 (e.g., water, saline, combinations thereof or other fluids).

In the embodiments shown herein, the probes of the present invention are typically illustrated to integrate plural members into a single probe wherein the plural members are configured for guiding light, providing sonic energy, delivering fluid or a combination thereof. It should be understood, however, that these members may be divided amongst multiple probes if desired. It should be further understood that the probe of the present invention may integrate members according to a variety of configurations within the scope of the present invention.

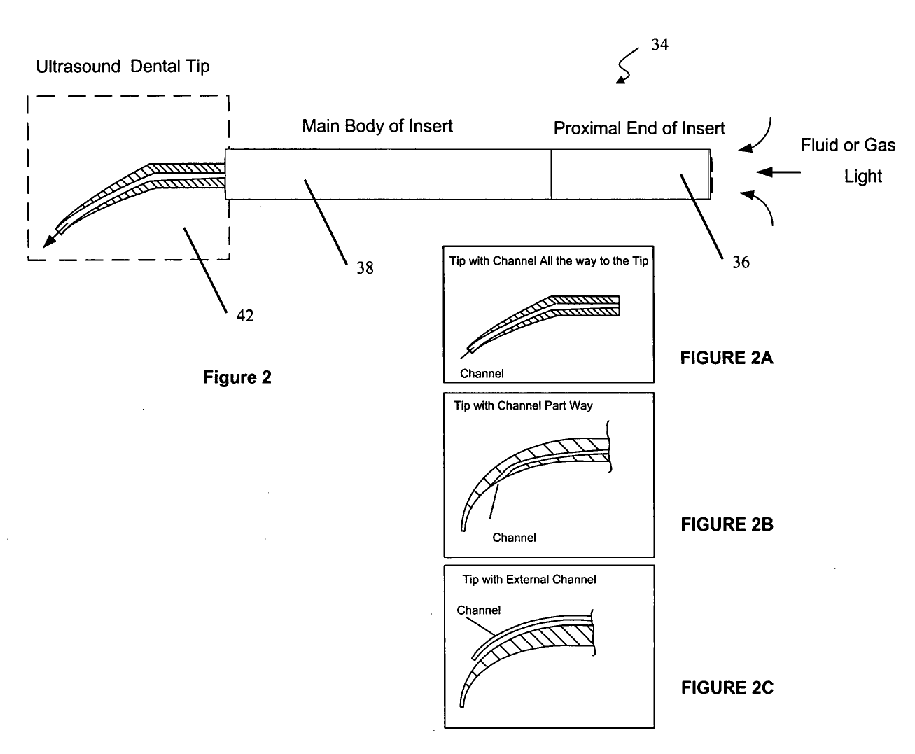

The probe 12 of FIG. 1 is shown in more detail in FIGS. 2-5. In the embodiment shown, the probe 12 includes an attachment shown as an insert 34 and the insert 34 includes a member for providing sonic (e.g., ultrasonic) energy, which is shown as a dental scaler tip 42. The insert 34 also includes a member for providing fluid, which is illustrated as a tube 44 and a member (e.g., a waveguide) for providing light, with is shown as an optical fiber 46.

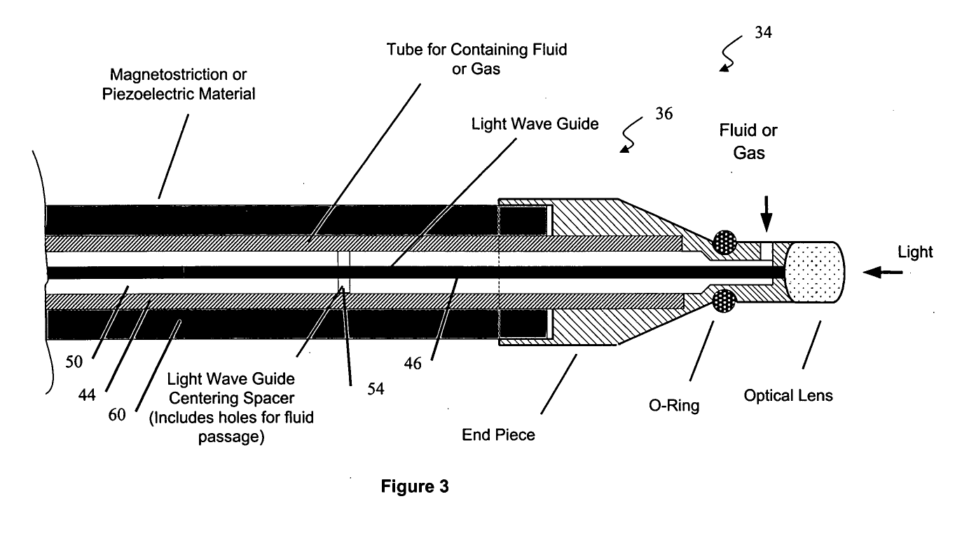

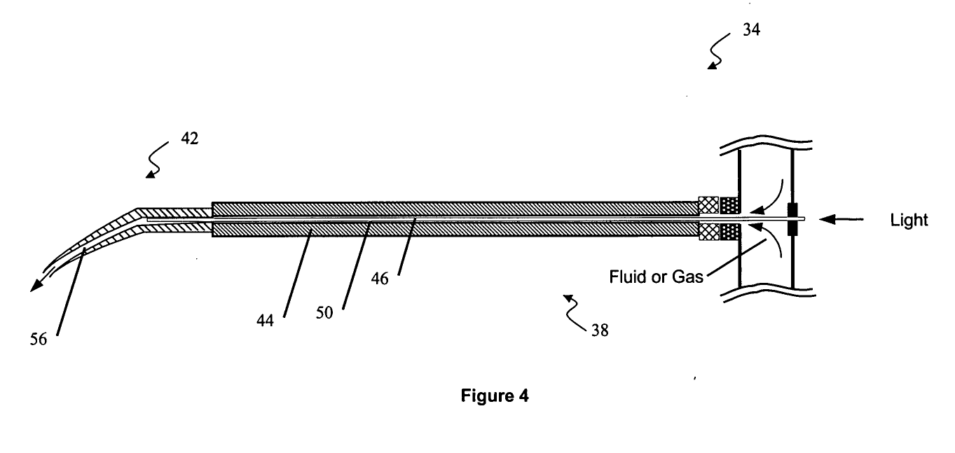

With reference to FIG. 2, the insert 34 is divided into a proximal portion 36 opposite the dental scaler 42 and a body portion 38 located between the proximal portion 36 and the dental scaler 42. FIG. 3 then illustrates the proximal portion 36 in greater detail while FIG. 4 illustrates the body portion 38 and the dental scaler 42 in greater detail.

In the illustrated embodiment, the tube 44 extends centrally along substantially the entire insert 34, the probe 12 or both and substantially defines the body portion 38 of the insert 34. The tube 44 defines a passageway or tunnel 50 that also extends along substantially the entire insert 34, the probe 12 or both. Typically, the tube 44 is in fluid communication with therapeutic fluid source 26 via tubes or other members.

The optical fiber 46 is located within the tunnel 50 and is substantially coextensive with the tube 44. As shown in FIG. 3, one or more spacers 54 may be employed for positioning or spacing the fiber 46 away from the tube 44. When used, the spacers 54 typically include openings (e.g., cavities, through-holes or the like) for allowing fluid flow therethrough.

The scaler tip 42 is attached to the tube 44 at one end of the tube 44. The scaler tip 42 defines its own tunnel 56, which is preferably in fluid communication with the tunnel 50 of the tube 44. The scaler tip 42 is preferably arced or curved, although not required.

Various tips or members may be employed for delivery of sonic energy and the use of the various tips or members contemplates that fluids may be delivered by those tips or members or delivered adjacent the tips or members using a variety of passageways. It is contemplated that a tip or other member may include one hole or multiple holes (e.g., arranged radially) for delivery of light, fluid or both or a tip may be formed of a porous (e.g., a microporous) structure for the delivery of light, fluid or both. FIGS. 2A-2C illustrate some examples of alternative tips.

As shown in the example of FIG. 2A, a passageway or tunnel may extend to a distal end of a tip. Alternatively, as shown in the example of FIG. 2B, a passageway or tunnel may extend only a portion of the distance to a distal end of a tip. As yet another alternative, as shown in the example of FIG. 2C, a tubular member or multiple tubular members separate from a tip may be configured for fluid delivery.

It is contemplated that the skilled artisan may be able to employ a variety of sonic energy sources within the scope of the present invention. Typically, the sonic energy source 20 includes an actuator material that assist in the creation and/or transmission of sonic energy to the member (e.g., the scaler tip) configured for delivery of the sonic energy and an activator for activating the actuator material. As an example, the sonic energy source could comprise a piezoelectric material in electrical communication with an electrical energy source wherein the piezoelectric material converts energy from the electric energy source into ultrasonic vibrations deliverable by a member such as the scaler tip. In particular, the piezoelectric material may deform or vibrate in response to the application of an electrical field at an ultrasonic frequency.

Generally, the actuator material may be configured in variety of shapes, sizes or other configurations. For example, the material could extend down the center of the probe and fluid delivery openings or other components of the probe may be outside the actuator material. Alternatively, the actuator material could comprise a plurality of rods and may or may not be tubular in configuration.

In the embodiment shown, there is an actuator material 60 integrated into the proximal portion 36 of the insert 34. The material 60 has a tubular configuration and substantially surrounds a portion of the tube 44 and a portion of the waveguide or fiber 46 of the insert. The particular actuator material 60 illustrated is a magnetostriction material that converts energy from an electric energy source 62 into ultrasonic vibrations deliverable by a member such as the scaler tip.

In the particular embodiment shown, the electrical energy source 62 includes excitation drive circuitry 64 configured for communicating the electrical energy from the electrical energy source 62 to the actuator material 60. In turn, the electrical energy exposes the actuator material 60 to a magnetic field that excites and vibrates the actuator material 60, which sonically or ultrasonically vibrates the tube 44 the scaler tip 42 or both. It is contemplated that the actuator material may be directly or indirectly connected to the member or tip for initiating the vibration.

Preferably, the apparatus 10 includes a controller 70 in signaling communication with the fluid sources 24, 26, 28 the light source 22 and the sonic energy source 20. The controller 70 will typically allow a user of the apparatus 10 to control the delivery of fluids, the delivery of light, the delivery and frequency of ultrasonic vibrations of the actuator material 60, the member or scaler tip 42, or both by the probe 12. The apparatus 10 can also include an activation device or switch 72 (e.g., an on/off foot controlled switch) for allowing the user to determine when ultrasonic vibrations, fluid, light or a combination thereof are to be delivered. It will be understood that a variety of different controllers and switches can be developed for controlling the probe and other components of the apparatus 10 within the scope of the present invention and depending upon the degree and type of control desired.

In the particular embodiment illustrated, the activation device 72 (e.g., switch or the like) can be linked to the excitation drive circuitry 64 and/or the control circuitry and can be used to control (1) the activation of electrical excitation to the sonic source 20 producing sonic energy; (2) the activation of light from the light source 22; and (3) the flow of fluid(s) (e.g., liquid, photosensitizing composition, gas) from the fluid sources 24, 26, 28 to the probe 12 or a combination thereof. The excitation drive circuitry 64 can also be configured for controlling amplitude of the electrical excitation to the sonic source 20, the light source, as well as the flow rate of fluid(s) to the probe 12. Fluid communication tubes 78 are connected and controlled by a switching device 80. The switching device 80 determines which of the fluid sources 24, 26, 28 (e.g., the liquid source 28, the therapeutic source 26 or the gas source 24) is delivered to the probe 12 via the tubes 78 and can be controlled by the controller 70. The switching device 80 can be any art-disclosed switching device and it can be optionally incorporated into the excitation drive circuitry 64. Thus the switching device 80 can comprise a single switch or solenoid in communication with two or all of the fluid sources, multiple switches or solenoids in connection with respective fluid sources or the like. Moreover, it is possible to have the switching device at least assist in controlling fluid flow rates.

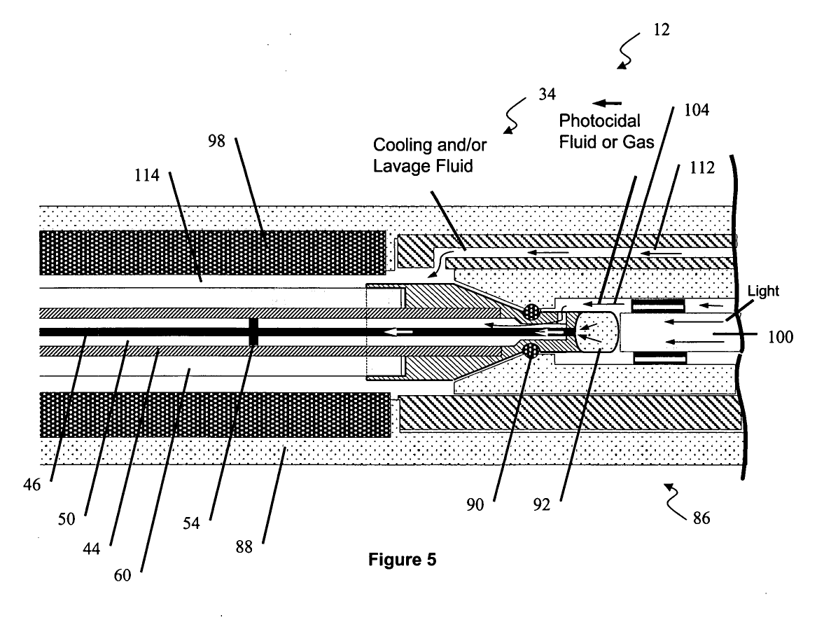

In FIG. 5, the insert 34 is connected to or placed in communication with the light source 22, the fluid sources 24, 26, 28 and the sonic energy source 20 with a connector 86 that is located within a housing 88 of the probe 12. In the embodiment shown, an end of the proximal portion 36 of the insert 34 is inserted within a seal 90 (e.g. an O-ring) for positioning the insert 34 relative to the connector 86. The end of the proximal portion 36 is illustrated to include an optional optical element 92 (e.g., a lens, a tapered member, a holographic element, an index matching element or the like) for assisting in coupling light between the source 100 and the fiber 46. Moreover, the housing includes an electrically conductive material 98 that can expose the actuator material 60 to an electric field, a magnetic field or both.

Advantageously, the insert 34 can be removed from the housing and cleaned and sterilized between uses.

### ii. Operation of the Apparatus

In use, the therapeutic fluid source delivers the fluid to the member configured for dispensing the fluid to an area of tissue. Thereafter, the light source communicates electromagnetic radiation to the member configured for delivering light to an area of tissue. Additionally, and typically at a close proximity in time to delivery of the photosensitizing composition or delivery of the light, the sonic energy source provides sonic energy to the member configured for delivering that sonic energy to an area of tissue. It should be understood that the areas of tissue to which the sonic energy, the fluid and the light are delivered are typically one single area of tissue, but such areas may be merely adjacent each other or only partially overlapping as well.

With reference to FIGS. 1-5, light is communicated from the light source 22 (e.g., a laser source) along a first waveguide 100 to the waveguide or optical fiber 46 of the probe 12, which guides the light to the tip 42 where it is emitted for delivery to an area of tissue. In the particular embodiment shown, the light exits the waveguide 100 within the connector 86 and enters the lens 92, which focuses the light into the waveguide or fiber 46 of the probe 12.

Photosensitizing composition flows from its source 26 through a tube 78 and passage 104 of the connector 86 to and through the tunnel 50 of the tube 44 of the probe 12 for delivery to an area of the tissue. In the particular embodiment shown, the fluid flows from the passage 104 to and through the opening 56 in the member or tip 42 of the probe 12.

In an alternative embodiment, it is contemplated that a member such as a tube may be connected to the source of therapeutic fluid and may be separate from the members used for delivery of light and/or sonic energy. In such an embodiment, the therapeutic fluid may be applied to tissue and then a probe including both a waveguide and a sonic scaling tip may be employed to provide light and sonic energy to the tissue.

In the illustrated embodiment, electrical energy is typically provided via a bus 110 (e.g., a wire or other electrical conductor) to the electrically conductive material 98, which in turn creates a magnetic field for exciting the actuator material 60. The actuator material then vibrates at an ultrasonic frequency and, in turn, vibrates the scaler tip 42 at an ultrasonic frequency.

It is additionally contemplated that the apparatus 10 may include a source 28 of cooling and/or lavage fluid (e.g., coupling fluid, water or saline) that can flow the fluid to and through the probe for delivery of the fluid to an area of tissue. In the particular embodiment shown, fluid is delivered through a tube 78 and a passage 112 in the connector and is delivered to a passage 114 defined within the probe 12 between the conductive material 98 and the actuator material 60. The fluid is then delivered to the scaler tip 42 and emitted to the area of tissue. It is particularly preferred, but not required, for the sonic energy to be provided to the tissue in the presence of such cooling and/or lavage fluid.

It is additionally contemplated that the cooling and/or lavage fluid, the photosensitizing composition or both may include one or more additives, which can provide therapeutic advantages. For example, the cooling and/or lavage fluid, the photosensitizing composition or both may include bubbles (e.g., microbubbles) trapped in shells for enhancing acoustic cavitation, sonoluminescence or both when the probe is used to perform sonophotodynamic therapy. These bubbles can be produced using art-disclosed means such as the use of hydrocarbons, fluorcarbons, perfluorochemicals, sulfur hexafluoride etc. The addition of bubbles with gas in them (e.g., air, nitrogen, helium, argon, xenon, or the like) has been reported to emit light at higher intensity during sonoluminescence. The size of the bubbles is optimized to have a natural resonance at the frequency of sonic energy employed. The frequency resonance of a gas bubble (fr) is known to be approximately related by the following equation: fr=(3gPo/r)^{1/2}/(2πa) where: g=the ratio of specific heats for a bas bubble, Po=ambient hydrostatic pressure, r=density of the surrounding media, and a=radium of the bubble in meters. Producing acoustic cavitation and sonoluminescence with lower applied acoustic intensity (e.g., tip vibration in the KHz ranges) is generally desired because of the potential problems with high intensity acoustic energy and non desired tissue effects.

It is also contemplated that gas (e.g., air, nitrogen, helium, argon, xenon, or the like) may be provided from the gas source 24 to any of the tunnels, openings, passageways or the like for purging or other purposes.

As suggested, the system apparatus 10 of the present invention may be employed for performing sonophotodynamic therapy upon a variety of tissue of nearly any organism, but that, the apparatus is particularly suited for performing dental sonophotodynamic therapy.

### iii. Alternative Embodiments

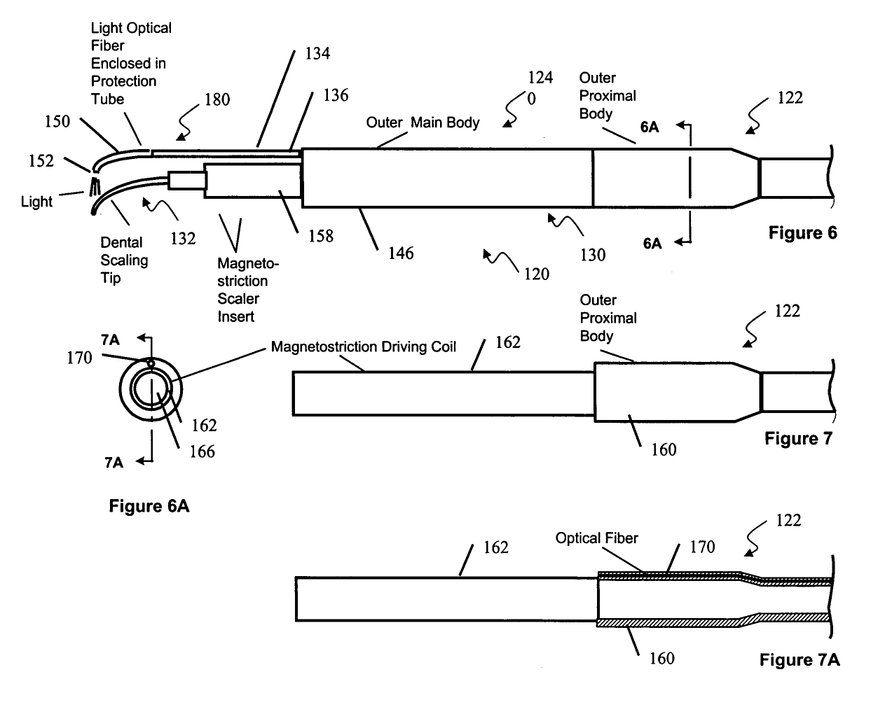

As suggested previously, the members and other components of the apparatus of the present invention may be arranged, integrated and connected to each according to a variety of protocols within the present invention. As such, FIGS. 6-9A illustrate alternative embodiments of probes suitable for use with the apparatus of the present invention. As the skilled artisan will recognize, the members and components have similarities in structure and use as compared to previous embodiments. As such, only differences are typically discussed, however, previous descriptions of similar or same components and uses thereof apply to the following embodiments as well.

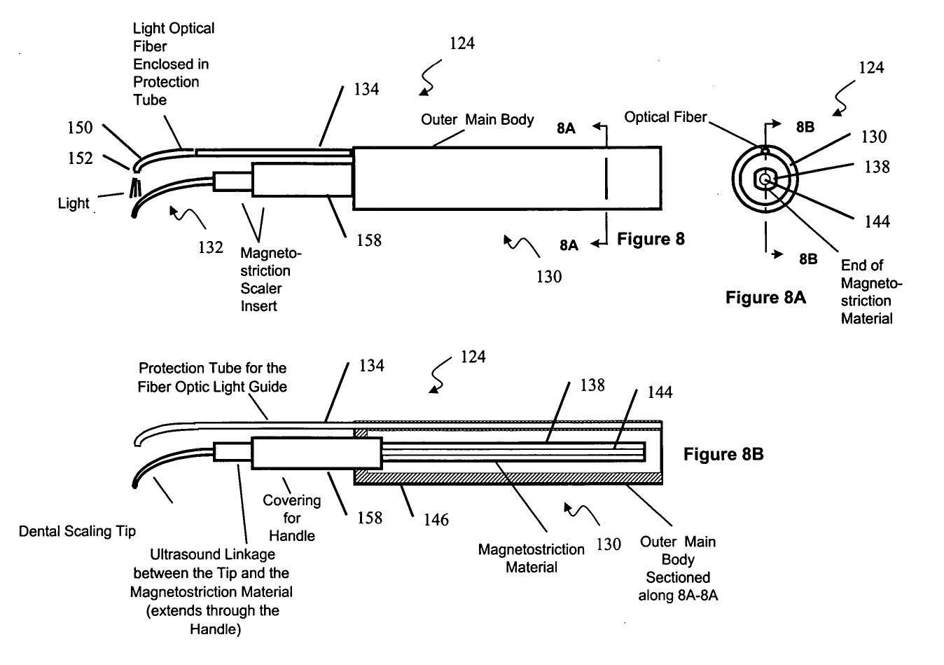

In FIGS. 6-8A, there is illustrated a probe 120 having a base or proximal portion 122 and an attachment 124 that attaches to the base portion 122. Referring to FIGS. 8 and 8A, the attachment 124 includes a housing portion 130, a member shown as a scaling tip 132 for delivery of ultrasonic energy and a section 134 of a member shown as an optical fiber 136 for delivery of light. The attachment 124 has an actuator material 138 located within and substantially coextensive with the housing portion 130.

The probe 120 preferably includes a member such as a tube 144 for delivering photosensitizing composition to and through the scaling tip 132. The optical fiber 136 is located within and extends along a wall 146 of the housing portion 130. As such the fiber 136 is substantially coextensive with the actuator material 138. In the embodiment shown, the fiber 136 extends outward from the housing portion 130 and is arced to emit light toward the scaling tip 132. It may be desirable to provide a protective encasing 150 about at least the end 152 of the section 134 of fiber 136. The attachment 124 is also shown to include a covering 158 for protecting a linkage portion that connects the actuator material 138 to the scaler tip 132.

With reference to FIGS. 6, 7 and 7A, the base portion 122 of the probe 120 includes a housing portion 160 and an electrically conductive material 162 (e.g., a magnetostriction driving coil) extending therefrom. The conductive material 162 is generally circular for defining a hollow portion 166 within the material 162. The housing portion 160 includes a section 170 of waveguide shown as optical fiber.

Upon attachment of the attachment portion 124 to the base portion 122, the section 134 of waveguide of the attachment portion 124 aligns with the section 170 of waveguide of the base portion 122 such that light can be transmitted down the lengths of the sections to the end 152 of the member or completed waveguide 180. Also upon attachment, the actuator material 138 is located in the hollow portion 166 of the conductive material 162 such that the actuator material 138 may be sonically vibrated as previously described.

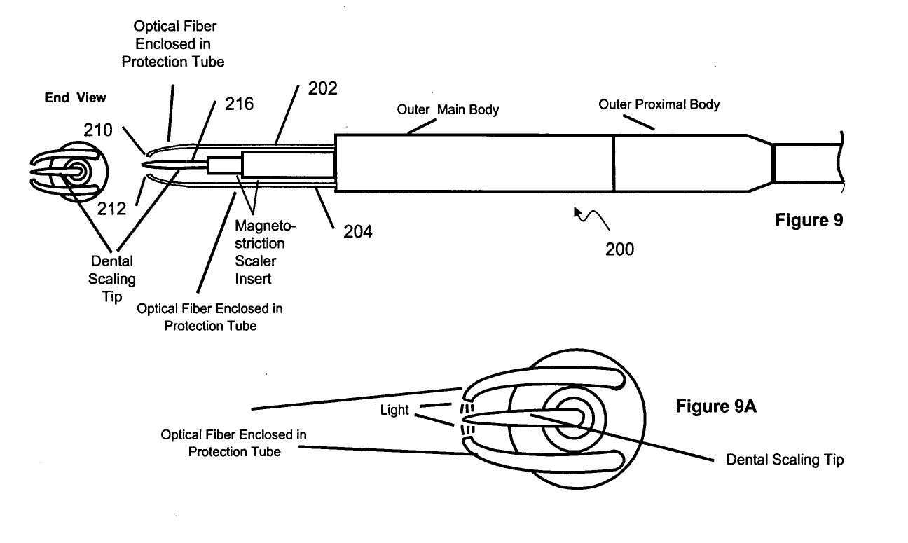

In another alternative embodiment and with reference to FIGS. 9 and 9A, a probe 200 similar to the probe 120 of FIGS. 6-8A is illustrated with the exception that the probe 200 includes two waveguides 202, 204. As shown, the waveguides 202, 204 are on opposite sides of the probe 200 and have ends 210, 212 that emit light in generally opposite directions, but both toward a scaling tip 216 of the probe 200. It will be understood that, at least in one embodiment, each of the waveguides 202, 204 could be configured similar to the waveguide 180 of FIGS. 6-8A and that additional waveguides or fibers could be added to the probe in a similar manner.

It is additionally contemplated that any of the fluids may be separately delivered rather than through the probe. For example, a syringe or a tube and pump assembly may be employed to deliver photosensitizing composition, cooling or lavage fluid or air or other gasses and then the probe may be used at the location of delivery of the fluid.

Unless stated otherwise, dimensions and geometries of the various structures depicted herein are not intended to be restrictive of the invention, and other dimensions or geometries are possible. Plural structural components can be provided by a single integrated structure. Alternatively, a single integrated structure might be divided into separate plural components. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention.

What is claimed is:

1. An apparatus for performing sonophotodynamic therapy, comprising:
a light source;
a sonic energy source;
a cooling or lavage fluid source; and
one or more members in communication with the light source, the sonic energy source and the cooling or lavage fluid source for respectively providing light, sonic energy and cooling or lavage fluid to tissue of an organism;
wherein the one or more members include a single member or a plurality of members integrated together.

2. An apparatus as in claim 1 further comprising a photosensitizing composition source in communication with the one or more members for providing photosensitizing composition to the tissue of the organism.

3. An apparatus as in claim 1 wherein the sonic energy source is configured for delivery of ultrasonic vibrations.

4. An apparatus as in claim 1 wherein the one or more members are integrated into a probe.

5. An apparatus as in claim 1 wherein the one or more members includes a waveguide in communication with the light source.

6. An apparatus as in claim 4 wherein the waveguide includes an optical fiber.

7. An apparatus as in claim 1 wherein the one or more members include a scaling tip in communication with the sonic energy source.

8. An apparatus as in claim 1 wherein the one or more members includes a tube in fluid communication with the photosensitizing composition source.

9. An apparatus as in claim 1 further comprising a source of pressurized gas in communication with the one or more members.

10. An apparatus as in claim 1 the sonic energy source includes an actuator material selected from a magnetostriction material or a piezoelectric material.

11. An apparatus for performing sonophotodynamic therapy, comprising:
a light source;
a sonic energy source;
a photosensitizing composition source; and
one or more members in communication with the light source, the sonic energy source and the photosensitizing composition source for respectively providing light, sonic energy and cooling or lavage fluid to tissue of an organism;
wherein the one or more members include a plurality of members integrated together; and
wherein the one or more members include a tube in fluid communication with the photosensitizing composition and a waveguide in communication with the light source, the waveguide being located at least partially within the tube.

12. An apparatus as in claim 11 wherein the light source is a laser source.

13. An apparatus as in claim 11 further comprising a cooling or lavage fluid source in communication wherein the one or more members are in fluid communication with the cooling or lavage fluid source.

14. An apparatus as in claim 11 wherein the sonic energy source is an ultrasonic energy source.

15. An apparatus as in claim 11 wherein at least one of the one or more members is part of an insert that can be removably integrated into a probe.

16. An apparatus as in claim 11 further comprising a source of pressurized gas in communication with the one or more members.

17. An apparatus as in claim 11 wherein the one or more members includes a waveguide for guiding the light from the light source.

18. An apparatus for performing sonophotodynamic therapy, comprising:
a light source;
a sonic energy source;
a photosensitizing composition source;
a cooling or lavage fluid source; and
one or more members in communication with the light source, the sonic energy source, the cooling or lavage fluid source and the photosensitizing composition source for providing ultrasonic energy, light and photosensitizing composition to tissue of an organism, wherein:
i. the one or more members include a waveguide for the delivery of light from the light source, a dental scaler for delivery of sonic energy from the sonic energy source, a tube defining a passageway for delivery of the cooling or lavage fluid from the cooling or lavage fluid source and a tube defining a passageway for delivery of photosensitizing composition from the photosensitizing composition source; and
wherein the one or more members include a plurality of members integrated together into a single probe.

19. An apparatus as in claim 18 wherein the waveguide is located within the passageway of the tube for the photosensitizing composition.

20. An apparatus as in claim 18 wherein the probe includes a housing and the waveguide is located within a wall of the housing.
